(19) United States
(12) Patent Application Publication
(10) Pub. No.: US 2022/0370958 A1
(43) Pub. Date: Nov. 24, 2022

(54) ANTIBODY CONJUGATED NANOPARTICLE ASSAY AND TREATMENT FOR SARS-CoV-2

(71) Applicants: Arizona Board of Regents on behalf a Arizona State University, Scottsdale, AZ (US); Halberd Corporation, Jackson Center, PA (US)

(72) Inventors: Qiang Chen, Chandler, AZ (US); Haiyan Sun, Chandler, AZ (US); Mitchell Steven Felder, Hermitage, PA (US)

(21) Appl. No.: 17/747,322

(22) Filed: May 18, 2022

Related U.S. Application Data
(60) Provisional application No. 63/190,398, filed on May 19, 2021.

(51) Int. Cl. B01D 57/00; A61K 35/14; A61K 35/24; A61K 35/38

(52) U.S. Cl. CPC B01D 57/00; A61K 35/14; A61K 35/24; A61K 35/38

(57) ABSTRACT

An embodiment provides a method for treatment of a viral antigen for the COVID-19 virus, including: obtaining a body fluid from a patient; introducing the body fluid to at least one binding antibody, wherein the at least one binding antibody binds to an antigen of the SARS-CoV-2 spike (S) protein and comprises a conjugated metal; forming a viral antigen-antibody complex; and removing the viral antigen-antibody complex from the body fluid using a radiofrequency method; and returning the body fluid to the patient. Other aspects are described and claimed.

### CROSS REFERENCE TO RELATED APPLICATION

This application claims priority to U.S. Provisional Patent Application Ser. No. 63/190,398, filed on May 19, 2021, and entitled “ANTIBODY CONJUGATED NANOPARTICLE ASSAY AND TREATMENT FOR SARS-CoV-2,” the contents of which are incorporated by reference herein.

### FIELD

This application relates generally to an assay and treatment for SARS-CoV-2, and, more particularly, to detection and removal of SARS-CoV-2 using a conjugated nanoparticle and extracorporeal radiofrequency technique.

### BACKGROUND

The instant application contains a Sequence Listing which has been submitted electronically in ASCII format and is hereby incorporated by reference in its entirety. Said ASCII copy, created on May 18, 2021, is named 995_018_P_SL.txt and is 7 KB in size.

This application relates generally to a treatment for SARS-CoV-2, and, more particularly, to a conjugation of antibodies with nanoparticles for treatment of a patient for SARS-CoV-2.

Coronaviruses represent a group of viruses that may lead to respiratory tract infections. These infections may range from mild to lethal. Coronaviruses may cause severe acute respiratory syndrome (SARS) and Middle East respiratory syndrome (MERS). A novel coronavirus (COVID-19) has led to a global pandemic causing a public health and economic crisis. Severe acute respiratory syndrome coronavirus 2 (SARS-CoV-2) is the strain of coronavirus that causes coronavirus disease 2019. Transmission may be through close contact of individuals and via respiratory droplets such as coughs or sneezes. Faster and more accurate methods of identifying and treating individuals infected with COVID-19 could mitigate the global pandemic.

### BRIEF SUMMARY

In summary, one embodiment provides a method for treatment of a viral antigen for the COVID-19 virus, comprising: obtaining a body fluid from a patient; introducing the body fluid to at least one binding antibody, wherein the at least one binding antibody binds to an antigen of the SARS-CoV-2 spike (S) protein and comprises a conjugated metal; forming a viral antigen-antibody complex; and removing the viral antigen-antibody complex from the body fluid using a radiofrequency method; and returning the body fluid to the patient.

Another embodiment provides a method for treatment of a bacterial antigen of a bacteriological infection, comprising: obtaining a body fluid from a patient; introducing the body fluid to at least one binding antibody, wherein the at least one binding antibody binds to the bacterial antigen and comprises a conjugated metal; forming a bacterial antigen-antibody complex; and removing the bacterial antigen-antibody complex from the body fluid using a radiofrequency method; and returning the body fluid to the patient.

A further embodiment provides a method for treatment of a viral antigen for the COVID-19 virus, comprising: obtaining a body fluid from a patient; introducing the body fluid to at least one binding antibody, wherein the at least one binding antibody binds to an antigen of the SARS-CoV-2 spike (S) protein and comprises a conjugated metal, wherein the conjugated metal is gold; forming a viral antigen-antibody complex; and removing the viral antigen-antibody complex from the body fluid using a radiofrequency method wherein the radiofrequency method comprises application of a radiofrequency field generated between a transmission head and a reception head generating heat surrounding the conjugated metal annihilating a disease causing potential; and returning the body fluid to the patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example method for a metal conjugated monoclonal antibody for the treatment of a viral or bacterial antigen.

FIG. 2 illustrates an example blot of gold particle conjugation with Anti-SARS-2 antibody.

FIG. 3 illustrates an example blot of gold particle conjugation with Anti-E. coli antibody.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a rapid and accurate assay of COVID-19 antibodies in a patient. For example, a patient may be tested to see if a vaccine or titer is necessary.

Accordingly, an embodiment provides a method for removal and/or determining the presence of a pathogen in body fluid of a patient using an extracorporeal radiofrequency technique. The pathogen may be the spike protein of SARS-CoV-2 or another region of COVID-19, a bacterial, a virus, or the like. The treatment may be from a patient's body fluid. The body fluid may be blood, CSF (cerebrospinal fluid), mucus, saliva, or any bodily fluid. The body fluid may be from a patient which may contain COVID-19 antibodies. In an embodiment, the body fluid may be exposed to at least one binding antibody. The antibody may be conjugated with a metal. The metal may be a particle or nanoparticle. The metal may be iron, gold, or the like. In an embodiment, a treatment may be applied to the body fluid. In an embodiment, a treatment may comprise exposing the body fluid to a binding antibody. The binding is to an antigen specific to the spike protein of SARS-CoV-2 or another region of COVID-19. In an embodiment, the method may determine the presence or absence of the antigen-antibody complex. The antigen-antibody complex may be destroyed or removed using a radiofrequency technique. The treated body fluid may be returned to a patient's body.

The illustrated example embodiments will be best understood by reference to the figures. The following description is intended only by way of example, and simply illustrates certain example embodiments.

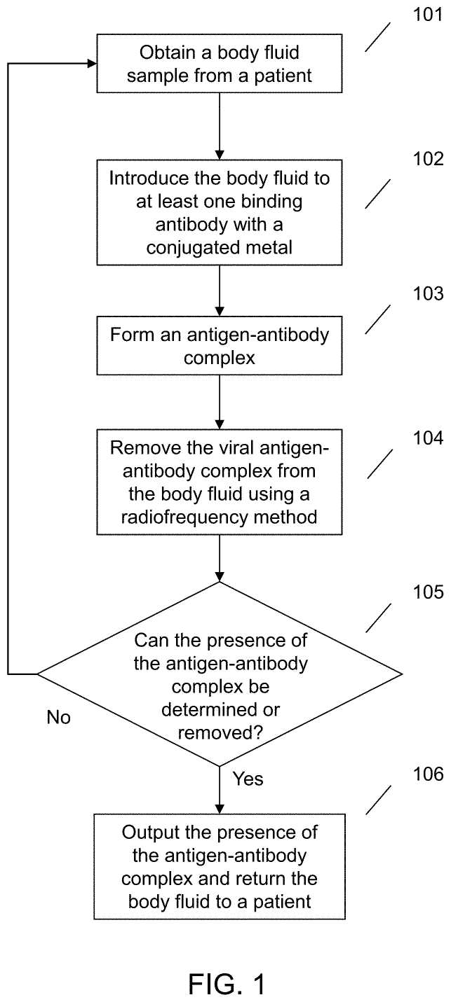

Referring to FIG. 1, an example device and method for conjugating a metal to an antibody. The antibody may be directed to a portion of the SARS-2 spike protein of COVID, a pathogenic virus, or the like. The body fluid may contain an antigen or analyte specific for the presence of COVID-19 antibodies or other viral or bacterial pathogen within the patient. The method may use a monoclonal antibody. The antibody may be directed to the spike protein of SARS-CoV-2 or another region of COVID-19. The antibody may have a fluorescent tag. The antibody may be conjugated with a metal. The metal may be a nanoparticle. The metal may be iron, gold, or the like. In an embodiment, the body fluid may be exposed to at least one binding antibody, form an antigen-antibody complex. The system and method may determine the presence of the antigen-binding antibody complex. The method and system may be used to determine if a patient has antibodies for the COVID-19 disease. It should be understood that the method and system described herein may be used for diagnostic purposes. In other words, the method may be used as a test kit for example at a medical facility, a testing facility, at home, or the like. The test may be a laminar flow assay. The method may use a conjugated metal antibody such the level of metal correlates to a level of antibody in the patient body. Conjugation may be tested using light absorption, observing migration on a molecular weight gel, or the like. Metal conjugated antibodies may be separated and or the antigen destroyed using radiofrequency or light energy techniques. Thus, the method may test for the presence of COVID-19, viral, or bacterial pathogen and/or treat a body fluid outside the patient body prior to returning the fluid to a patient.

At 101, in an embodiment, a method may identify, detect, or extracorporeally treat COVID-19, viral, or bacterial pathogen. The identification may be rapid in time. The identification may be from a patient's body fluid. The body fluid may be blood, CSF (cerebrospinal fluid), mucus, saliva, or any bodily fluid. The body fluid may be from a patient which may contain COVID-19 antigen, analyte, virions, or the like. An analyte or antigen within the body fluid may be a measure of an antibody protection of a patient. An analyte or antigen may be able to bind to a monoclonal antibody selective for the analyte.

For example, a sample or a body fluid may be withdrawn from a patient using standard medical techniques. Techniques may include sterile cotton swab, blood draw, lumbar puncture, or another accepted form of body fluid collection. Collection may include methodology to preserve the sample such as temperature regulation, sterile techniques, stability agents, buffers, or the like.

At 102, in an embodiment, the body fluid may be exposed to at least one binding antibody. For example, the antibody may be a monoclonal antibody and may selectively bind the spike protein of SARS-CoV-2 or another region of COVID-19. Additionally or alternatively, the antibody may bind to a viral or bacteriological pathogen antigen. The antibody may be conjugated with a metal or nanoparticle. The metal may be iron, gold, or the like.

At 103, in an embodiment, the COVID-19 antigen or analyte present in the body fluid may form an antigen-antibody complex with the binding antibody. In an embodiment, detection of viral antigen may be performed using a monoclonal antibody. Alternatively, a method for the detection of immunity may be performed using a secondary antibody which may binds to a primary antibody and antigen in the patient fluid.

In an embodiment, the antibodies listed below may be used:

Antibody B16, Mus musculus VH nucleotide sequence:
CAAGTACAGCTGCAGGAGTCTGGACCTGAGCTGGTGAAGCCTGGGGCTTT
AGTGAAGATATCCTGCAAGGCTTCTGGTTACACCTTCACAACCTACGATA
TAAACTGGATGAAGCAGAGGCCTGGACAGGGACTTGAGTGGATTGGATGG
ATTTATCCTGGAGATGGGAGTACAAAGTACAATGAGAAATTCAGGGGCAA
GGTCACACTGACTGCAGACAAATCCTCCAACACAGTCTACATGCACCTCA
TCAGCCTGCCTTCTGAGAAGTCTGCAGTCTATTTCTGTGCAAGATCGGTC
CTGGGACGGGGGTTTACTTACTGGGGCCAAGGGACTCTGGTCACTGTCTC
TGCAG, with an amino acid sequence: QVQLQESGPELVKP
GALVKISCKASGYTFTTYDINWMKQRPGQGLEWIGWIYPGDGSTKYNEKF
RGKVTLTADKSSNTVYMHLISLPSEKSAVYFCARSVLGRGFTYWGQGTLV
TVSA.
Antibody B16, Mus musculus VL nucleotide sequence:
GACATTGTGATGACACAGACTCCAGCTTCTTTGGCTGTGTCTCTAGGGCA
GAGGGCCACCATATCCTGCAGAGCCAGTGAAAGTGTTGATAGTTATGGCA
ATAGTTTTATGCACTGGTACCAGCAGAAACCAGGACAGCCACCCAAAGTC
CTCATCTATTTTGCATCCAACCTAGAATCTGGGGTCCCTGCCAGGTTCAG
TGGCAGTGGGTCTAGGACAGACTTCACCCTCACCATTGATCCTGTGGAGG
CTGATGATGCTGCAACCTATTACTGTCAGCAAAATAATGAGGATCCATAC

ACGTTCGGAGGGGGGACCAAGCTGGAAATAAAAC, with an amino
acid sequence: DIVMTQTPASLAVSLGQRATISCRASESVDSYGNS
FMHWYQQKPGQPPKVLIYFASNLESGVPARFSGSGSRTDFTLTIDPVEAD
DAATYYCQQNNEDPYTFGGGTKLEIK.
Antibody N12, Mus musculus VH nucleotide sequence:
CAAGTGCAGCTGGAGGAGTCTGGACCTGAGCTGGTGAAGCCTGGGGCTTT
AGTGAAGATATCCTGCAAGGCTTCTGGTTACACCTTCACAAGCTACGATA
TAAACTGGATGAAGCAGAGGCCTGGACAGGGACTTGAGTGGATTGGATGG
ATTTATCCTGGAGATGGTAGTACTAAGTACAATGAGAAATTCAAGGGCAA
GGCCACACTGACTGCAGACAAATCCTCCAGCACAGCCTACATGCAGATCA
GTAGCCTGACTTCTGAAAACTCTGCAGTCTATTTCTGTGCAAGATCCGAC
TTCGGCCACGGGTTTGTTTACTGGGGCCAAGGGACTCTGGTCACTGTCTC
TGCA, with an amino acid sequence: QVQLEESGPELVKPG
ALVKISCKASGYTFTSYDINWMKQRPGQGLEWIGWIYPGDGSTKYNEKFK
GKATLTADKSSSTAYMQISSLTSENSAVYFCARSDFGHGFVYWGQGTLVT
VSA.
Antibody N12, Mus musculus VL nucleotide sequence:
GATATTGTGCTCACACAGTCTCCAGCTTCTTTGGCTGTGTCTCTAGGGCA
GAGGGCCACCATATCCTGCAGAGCCAGTGAAAGTGTTGATACTTATGACA
ATAGTTTTATGCACTGGTACCAGCAGAAACCAGGACAGCCACCCAAACTC
CTCATCTATCTTGCATCCAACCTAGAATCTGGGGTCCCTGCCAGGTTCAG
TGGCAGTGGGTCTAGGACAGACTTCACCCTCACCATTGATCCTGTGGAGG
CTGATGATGCTGCAATCTATTACTGTCAGCAAAATTATGAGGATCCGTAC
ACGTTCGGAGGGGGGACCAAGCTGGAAATAAAAC, with an amino
acid sequence: DIVLTQSPASLAVSLGQRATISCRASESVDTYDNS
FMHWYQQKPGQPPKLLIYLASNLESGVPARFSGSGSRTDFTLTIDPVEAD
DAAIYYCQQNYEDPYTFGGGTKLEIK.

At 104, in an embodiment, the antigen-antibody complex may be removed from the body fluid. In an embodiment, the antigen-antibody complex may be quantified or measured. The removal may be referred to as a treatment. The treatment may be an extracorporeal radio frequency treatment. In an embodiment, a treatment may be applied to the body fluid. In an embodiment, a treatment may comprise exposing the body fluid to a binding antibody. The binding may be to an antigen specific to the spike protein of SARS-CoV-2. The antigen may include the of SARS-CoV-2 spike glycoprotein. Other antigens may be included for testing as well. Other antigens may include Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex forming antigen-antibody complexes, and combinations thereof. The binding antibody and Covid-19 specific antigen form an antigen-antibody complex.

In an embodiment, a treatment is applied to a body fluid extracorporeally. The treatment comprises exposing the body fluid to a tagged antibody generated to bind specific targeted pathogenic antigens (TPAs) of the Covid-19 virus, or other target, such as those described above. During this treatment the conjugated antibody(s) and the targeted pathogen antigen form antibody complexes. For example, the antibody may be conjugated with a metal or metal nanoparticle. The metal may be iron, gold, or the like. A method for enhancing radiofrequency (RF) absorption includes providing targeted RF enhancers, such as antibodies with an attached RF absorption enhancer, such as, for example metal particles. The antibodies target and bind to the Covid-19 virion. Binding RF enhancing particles to the antibodies (and other carriers having at least one targeting moiety) permits the injection of the antibodies (and other carriers having at least one targeting moiety) into the extracorporeal target solution. The RF enhancers induce the absorption of energy in the antibody-RF enhancing moiety complex. In addition, a combination of antibodies (and other carriers having at least one targeting moiety bound to different metals (and other RF absorbing particles) can be used allowing for variations in the RF absorption characteristics in the extracorporeal target area. The energy of the emitted radiofrequency (RF) annihilates the antibody-RF enhancing moiety complex, thereby destroying its disease-causing potential. The entire system is monitored and controlled utilizing a computer, in real time, utilizing time units of 1 millisecond or less during the entire procedure. Persons having ordinary skill in art will recognize that the steps described above can be performed on various devices/machines. This disclosure contemplates all known devices/machine that can perform the steps described in the above illustrative example.

A second stage substantially eliminates, through a high-energy radiofrequency emissive source targeting and annihilating, the antibody-RF enhancing moiety complex in the body fluid. A method for killing the Covid-19 virus, or other virus or bacteria, is by introducing into the extracorporeal patient body fluid (blood or CSF) RF absorption enhancers capable of selectively binding to the target and further capable of generating sufficient heat to kill or damage the bound target antigen-antibody complexes by heat generated solely by the application of an RF field generated by an RF signal between a transmission head and a reception head.

In an embodiment, the methodology described herein may be used to treat other conditions. For example, target antigens may be constructed for many conditions, diseases, infections, or the like. Pathogenic bacteria examples such as, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, Yersinia pseudotuberculosis, or the like may be treated.

At 105, in an embodiment, the method may determine the presence or absence of the antigen-antibody complex. The binding antibody may include, for example, a fluorescent antibody, a luminous antibody, a metal conjugated antibody, combinations thereof, or the like. The concentration of binding antibody may be made as high as necessary for the identification of extremely small, e.g., picogram/microliter, concentrations of the final antigen-antibody complex.

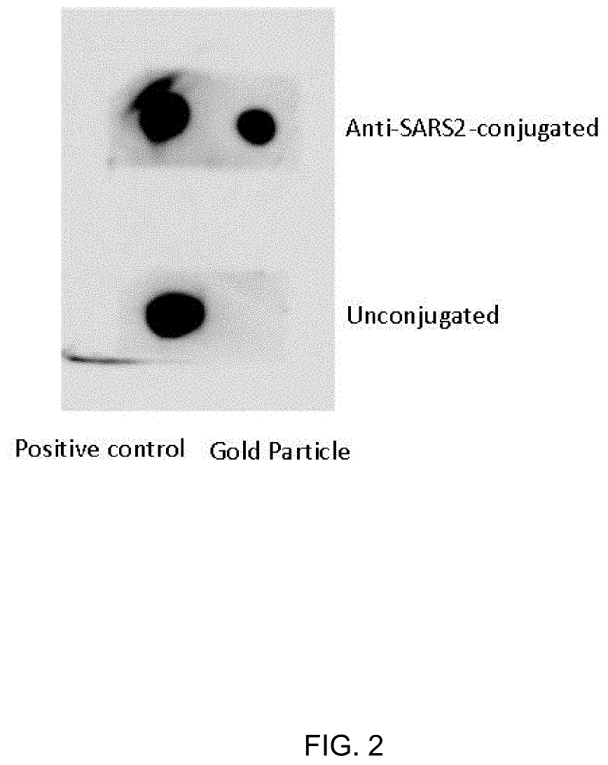

Example data for a metal conjugation with SARS-2 antibody spike protein are illustrated in FIG. 2. As an example, an antibody may be conjugated with a gold particle. For example, a gold particle of 20 nm is illustrated. In another embodiment the gold particles may be 40 nm diameter. Other gold particle diameters are contemplated and disclosed. The antibodies may be selected from those disclosed herein. The left column illustrates a positive control in which the spike protein was loaded onto the membrane. The positive control demonstrates the presence of antibody. The right column illustrates the spike protein binding in both anti-SARS2-conjugated (upper right) and unconjugated forms (lower right). The anti-SARS-conjugated was exposed and conjugated with gold particles demonstrating binding in the presence of the gold conjugation. The unconjugated sample comprises naked gold particles, and the lack of signal indicates there is no binding of the gold particles to the spike protein. Thus, unconjugated gold particles do not bind with the spike protein, and the antibody conjugated with the gold may bind the SARS-2 spike protein. The example demonstrates the use of gold particle conjugation for a SARS-2 spike protein, however, the method may be used for the treatment of other diseases such as bacterial pathogens.

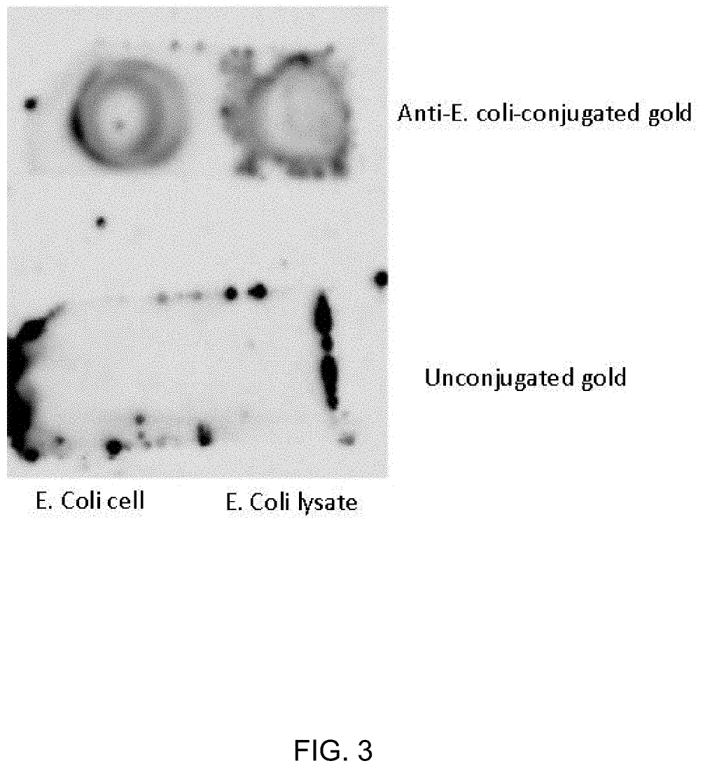

Example data for E. Coli and conjugated gold particles with E. Coli antibody are illustrated in FIG. 3. In an embodiment, the top and bottom membranes were coated with E. Coli cells (left), and E. Coli lysate (right, puréed). The unconjugated gold does not bind to the E. Coli, while the gold particles conjugated to the E. Coli antigen demonstrate binding to the E. Coli cells. Gold particle size may be selected as described above. The method may use iron or other metal for conjugation.

In an embodiment, the conjugated antibody may be treated using the RF method described above. The RF method may destroy or remove the antigen and disease causing portion to allow a return of the body fluid to a patient. Conjugation may be tested using various methods. For example, a conjugate particle has a different light absorption peak than the gold particle by itself. This shift in maximum absorption may indicate conjugation. Also, a focused bean of energy, such as light, may be used to eradicate a target bound by a metallic moiety as described herein. For example, the higher the shift in peak, the more conjugation. Another test may be to run a sample on a molecular gel and determine the migration. For example, a higher efficiency coating or conjugation creates a larger and slower migrating particle. For example, increased migration between the nanoparticle and the antibody, indicates an increase in size and a greater efficiency of coating. Unconjugated (surplus) antibody may need to be removed, which requires removal to reduce competition with the conjugated particles for binding with antigens. A proper centrifugation speed may separate conjugated and unconjugated antibody.

In an embodiment, a lateral flow device may be used. Although a metal conjugate may be the primary indicated, a fluorescent tag may be used. A body fluid or a portion of a body fluid may be added to a lateral flow device. In an embodiment, the body fluid may contain an antigen or analyte of COVID-19. The laminar flow device may contain reagents upon a surface. The antigen or analyte may flow via capillary action along a length of the laminar flow device. The laminar flow device may contain the COVID-10 specific antibodies described herein. These antibodies may be referred to as reporter antibodies. The reporter antibodies may migrate to a test line. The laminar flow device may also comprise a test line for a known analyte to confirm proper operation of the laminar flow test. In an embodiment, a Covid-19 antibody may comprise a fluorescent tag. In an embodiment, a fluorescence signal may correlate to the amount of COVID-19 immunity of a patient.

In an embodiment, the spike protein of SARS-CoV-2 antibody may contain an albumin moiety. This antibody may target and rapidly identify COVID-19 antigens. The antibody may or may not include a fluorescent tag. The fluorescent tag may be used for detection techniques. The fluorescent tag may be Alexa-488, Indocyanine green (ICG) or the like.

In an embodiment, as more antigens are present, the more antibodies will bind and increase the fluorescence signal. Therefore, this antibody may be used as a viral load gradient diagnostic assay. This may allow a physician to determine if a patient has sufficient antibodies, whether they have been vaccinated, if they need a booster, etc. This can be translated into a lateral flow type device. The test may provide a gradient measure of protection, not simply a go-no go test, as exists today. This could screen those who do not need the vaccine due to natural immunity, like those who are asymptomatic. Such a test may direct vaccine resources to a patient with a need for the vaccine.

Additionally or alternatively to lateral flow, the method may utilize different techniques for determining the presence or absence of the antigen-antibody complex. As an example, a dialysis or a variant of dialysis may be used. The dialysis may be used to remove the fluorescent antibody-antigen complex. This may allow for a rapid identification of a COVID-19 sample. Such technique may be automated, controlled by a computer system, or the like. The system may use a threshold, limits, alarms, or the like.

In an embodiment, the method may use flow cytometry analysis of fluorescent labelled antibodies relating to COVID-19. For example, flow cytometry analysis of fluorescent mAbs against SARS-CoV-2 spike (S) protein may be performed. For example, K562 cells may be fixed with 4% PFA (Paraformaldehyde) then permeabilized with 0.1% saponin in PBS (Phosphate-buffered saline). Cells may then be stained with anti-S mAb 1 or with mAb 1 that has been fluorescently labeled with Alexa488. Stained cells may be processed by flow cytometry. A rightward shift of fluorescent intensity indicates the fluorescent labeling of mAb 1.

In an embodiment, a method may utilize a designer fluorescent antibody with an attached macromolecular moiety. The macromolecular moiety, attached to the antibody, may be 1.000 mm to 0.00001 mm in diameter. Disclosed diameters are illustrative and may vary. The antibody-macromolecular moiety-targeted antigen complex would then be blocked for analysis, by using a series of microscreens which contain openings with a diameter 50.00000% to 99.99999% less than the diameter of the designer antibody-macromolecular moiety.

In an embodiment, methodology comprising the removal of the targeted antigen(s)/TA(s) by using a designer fluorescent antibody containing an iron (Fe) moiety. This will then create an Fe-fluorescent Antibody-Antigen (COVID-19/virion) complex. This iron containing complex may then be efficaciously removed using a strong, localized magnetic force field, which may be identified as positive. The conjugated metal may be a ferromagnetic metal. The conjugated metal may be a nanoparticle. The conjugated metal may have a gold coating.

In an embodiment, a variant of gel filtration chromatography, which may be utilized for the rapid identification of COVID-19. The fluorescent antibody-target antigen would be used to transport the sample through a size exclusion column that would be used to separate the fluorescent antibody-target antigen by size and molecular weight.

In an embodiment, a methodology using a molecular weight cutoff filtration may be employed. Molecular weight cut-off filtration refers to the molecular weight at which at least or approximately 80% of the target antigen(s)/TA(s) may be prohibited from membrane diffusion.

In an embodiment, a removal methodology for the fluorescent antibody-target antigen(s) may be used. The removal methodology may be selected from a group comprising a mechanical filter, a chemical filter, a dialysis machine, a molecular filter, molecular adsorbent recirculating system (MARS), a plasmapheresis unit, or combinations thereof.

For example, virions may be captured using antibody microarrays. The microarray may contain one or more binding antibody. In an embodiment, the binding antibody may comprise a fluorescent antibody (FI). In another embodiment, the binding antibody may comprise a luminescent (Lu) antibody. The microarray may comprise a plurality of antibodies fixed on a solid surface. The solid surface may be any suitable material. The microarray material may be transparent, such as glass, plastic, silicon, combinations thereof, or the like. The microarray may allow detection of at least one virion antibody complex. The microarray can comprise a plurality of monoclonal antibodies attached at high density on the solid surface. Typically, the microarray may contain millions of antibodies. Exposure of the virion to the binding antibodies on the microarray creates the virion. The complex may be tracked using an appropriate sensor. To identify the virion antibody complex after exposure in the microarrays, the body fluid may then be forced through a container preferably constructed from a transparent material, which exposes the virion antibody complex to a light-sensing device. The sensing device may also create an enlarged, magnified visual image of virion antibody complex. A concentrated and focused intense energy beam, such as light, is then used to properly illuminate the virion antibody complex within the body fluid. Each virion antibody complex may be rapidly identified and/or eradicated. The virion antibody complex may also be identified and tracked using optical or digital enhancement or magnification.

At 105, in an embodiment, if an antigen-antibody complex cannot be determined, the system may continue to determine the presence of another antigen-antibody complex in the body fluid. Alternatively, the method or system may determine that the patient body fluid does not contain the antigen or analyte. For example, the patient does not have COVID-19 and/or the patient may not have any immunity to COVID-19. Additionally or alternatively, the system may output an alarm, log an event, or the like. If an antigen-antibody complex can be determined, the system may provide an output at 106. If an antigen-antibody complex may be removed, for example using the RF method, the body fluid may be returned to a patient's body at 106. For example, CSF removed from a patient containing a virus or bacteria, may have a pathogen removed, and the CSF may be returned to the patient. Other types of body fluids may be treated as well. The antigen-antibody complex determination may be an output that is provided to a device in the form of a display, printing, storage, audio, haptic feedback, or the like. Alternatively, or additionally, the output may be sent to another device through wired, wireless, fiber optic, Bluetooth®, near field communication, or the like.

The various embodiments described herein thus represent a technical improvement to the detection of immunity or viral load directed to the spike protein of SARS-CoV-2 or other regions of SARS-CoV-2. Using the techniques as described herein, an embodiment may use a method to determine the presence or absence of SARS-CoV-2 in a sample from a patient.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treatment of a viral antigen for the COVID-19 virus, comprising:
obtaining a body fluid from a patient;
introducing the body fluid to at least one binding antibody, wherein the at least one binding antibody binds to an antigen of the SARS-CoV-2 spike (S) protein and comprises a conjugated metal;
forming a viral antigen-antibody complex; and
removing the viral antigen-antibody complex from the body fluid using a radiofrequency method; and
returning the body fluid to the patient.

2. The method of claim 1, wherein the at least one binding antibody comprises an isolated monoclonal antibody comprising a VH domain having the amino acid sequence of SEQ. ID No. 2 and a VL domain having the amino acid sequence of SEQ ID. No. 4.

3. The method of claim 1, wherein the at least one binding antibody comprises an isolated monoclonal antibody comprising a VH domain having the amino acid sequence of SEQ. ID No. 6 and a VL domain having the amino acid sequence of SEQ ID. No. 8.

4. The method of claim 1, wherein the body fluid is selected from the group consisting of: blood, cerebrospinal fluid, mucus, and saliva.

5. The method of claim 1, wherein the conjugated metal is a ferromagnetic metal with a gold coating.

6. The method of claim 1, wherein the conjugated metal is a radiofrequency absorption enhancer in which the radiofrequency method removes the viral antigen-antibody complex from the body fluid destroying a disease causing potential.

7. The method of claim 1, wherein the radiofrequency method comprises application of a radiofrequency field generated between a transmission head and a reception head.

8. The method of claim 1, further comprising eradicating the viral antigen-antibody complex using a light source.

9. The method of claim 1, further comprising at least one additional monoclonal antibody from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex forming antigen-antibody complexes.

10. The method of claim 1, further comprising at least one additional binding antibody which binds to an antigen of a pathogenic bacteria.

11. A method for treatment of a bacterial antigen of a bacteriological infection, comprising:
obtaining a body fluid from a patient;
introducing the body fluid to at least one binding antibody, wherein the at least one binding antibody binds to the bacterial antigen and comprises a conjugated metal;
forming a bacterial antigen-antibody complex; and
removing the bacterial antigen-antibody complex from the body fluid using a radiofrequency method; and
returning the body fluid to the patient.

12. The method of claim 11, further comprising determining an amount of the bacterial antigen-antibody complex in the body fluid.

13. The method of claim 11, wherein the at least one binding antibody further comprises a tag selected from the group consisting of: a fluorescent tag and a luminous tag

14. The method of claim 11, wherein the body fluid is selected from the group consisting of: blood, cerebrospinal fluid, mucus, and saliva.

15. The method of claim 11, wherein the conjugated metal is a ferromagnetic metal with a gold coating.

16. The method of claim 11, wherein the conjugated metal is a radiofrequency absorption enhancer in which the radiofrequency method removes the bacterial antigen-antibody complex from the body fluid destroying a disease causing potential.

17. The method of claim 11, wherein the radiofrequency method comprises application of a radiofrequency field generated between a transmission head and a reception head.

18. The method of claim 11, further comprising eradicating the bacterial antigen-antibody complex using a light source.

19. The method of claim 11, further comprising at least one additional monoclonal antibody from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex forming antigen-antibody complexes.

20. A method for treatment of a viral antigen for the COVID-19 virus, comprising:
obtaining a body fluid from a patient;
introducing the body fluid to at least one binding antibody, wherein the at least one binding antibody binds to an antigen of the SARS-CoV-2 spike (S) protein and comprises a conjugated metal, wherein the conjugated metal is gold;
forming a viral antigen-antibody complex; and
removing the viral antigen-antibody complex from the body fluid using a radiofrequency method wherein the radiofrequency method comprises application of a radiofrequency field generated between a transmission head and a reception head generating heat surrounding the conjugated metal annihilating a disease causing potential; and
returning the body fluid to the patient.


SEQUENCE LISTING (8 sequences)

SEQ ID NO: 1 - DNA, 355 bp, Mus musculus
caagtacagctgcaggagtctggacctgagctggtgaagcctggggctttagtgaagatatcctgcaaggcttctggttacaccttcacaacctacgatataaactggatgaagcagaggcctggacagggacttgagtggattggatggatttatcctggagatgggagtacaaagtacaatgagaaattcaggggcaaggtcacactgactgcagacaaatcctccaacacagtctacatgcacctcatcagcctgccttctgagaagtctgcagtctatttctgtgcaagatcggtcctgggacgggggtttacttactggggccaagggactctggtcactgtctctgcag

SEQ ID NO: 2 - protein, 118 aa, Mus musculus
QVQLQESGPELVKPGALVKISCKASGYTFTTYDINWMKQRPGQGLEWIGWIYPGDGSTKYNEKFRGKVTLTADKSSNTVYMHLISLPSEKSAVYFCARSVLGRGFTYWGQGTLVTVSA

SEQ ID NO: 3 - DNA, 334 bp, Mus musculus
gacattgtgatgacacagactccagcttctttggctgtgtctctagggcagagggccaccatatcctgcagagccagtgaaagtgttgatagttatggcaatagttttatgcactggtaccagcagaaaccaggacagccacccaaagtcctcatctattttgcatccaacctagaatctggggtccctgccaggttcagtggcagtgggtctaggacagacttcaccctcaccattgatcctgtggaggctgatgatgctgcaacctattactgtcagcaaaataatgaggatccatacacgttcggaggggggaccaagctggaaataaaac

SEQ ID NO: 4 - protein, 111 aa, Mus musculus
DIVMTQTPASLAVSLGQRATISCRASESVDSYGNSFMHWYQQKPGQPPKVLIYFASNLESGVPARFSGSGSRTDFTLTIDPVEADDAATYYCQQNNEDPYTFGGGTKLEIK

SEQ ID NO: 5 - DNA, 354 bp, Mus musculus
caagtgcagctggaggagtctggacctgagctggtgaagcctggggctttagtgaagatatcctgcaaggcttctggttacaccttcacaagctacgatataaactggatgaagcagaggcctggacagggacttgagtggattggatggatttatcctggagatggtagtactaagtacaatgagaaattcaagggcaaggccacactgactgcagacaaatcctccagcacagcctacatgcagatcagtagcctgacttctgaaaactctgcagtctatttctgtgcaagatccgacttcggccacgggtttgtttactggggccaagggactctggtcactgtctctgca

SEQ ID NO: 6 - protein, 118 aa, Mus musculus
QVQLEESGPELVKPGALVKISCKASGYTFTSYDINWMKQRPGQGLEWIGWIYPGDGSTKYNEKFKGKATLTADKSSSTAYMQISSLTSENSAVYFCARSDFGHGFVYWGQGTLVTVSA

SEQ ID NO: 7 - DNA, 334 bp, Mus musculus
gatattgtgctcacacagtctccagcttctttggctgtgtctctagggcagagggccaccatatcctgcagagccagtgaaagtgttgatacttatgacaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaatctattactgtcagcaaaattatgaggatccgtacacgttcggaggggggaccaagctggaaataaaac

SEQ ID NO: 8 - protein, 111 aa, Mus musculus
DIVLTQSPASLAVSLGQRATISCRASESVDTYDNSFMHWYQQKPGQPPKLLIYLASNLESGVPARFSGSGSRTDFTLTIDPVEADDAAIYYCQQNYEDPYTFGGGTKLEIK